(19) United States
(12) Patent Application Publication
(10) Pub. No.: US 2022/0347630 A1
(43) Pub. Date: Nov. 3, 2022

(54) SIMPLIFIED FLUID CONNECTION AND MEMBRANE FILTER

(71) Applicant: STOBBE GMBH, Morbio Inferiore (CH)

(72) Inventor: Per STOBBE, Morbio Inferiore (CH)

(73) Assignee: STOBBE GMBH, Morbio Inferiore (CH)

(21) Appl. No.: 17/766,069

(22) PCT Filed: Oct. 8, 2020
(86) PCT No.: PCT/EP2020/078275

§ 371 Date: Apr. 1, 2022

(30) Foreign Application Priority Data
Oct. 9, 2019 (DK) PA 2019 01194

(51) Int. Cl. B01D 63/02; B01D 61/20

(52) U.S. Cl. CPC B01D 63/02; B01D 63/021; B01D 61/20; B01D 2313/00; B01D 2313/10; B01D 2313/12; B01D 2313/13; B01D 2313/20; B01D 2313/21; B01D 2313/28; B01D 2313/04; B01D 2313/44; B01D 2313/2061; B01D 2315/10

(57) ABSTRACT

The invention concerns a set of tubular elastic coupling bodies arranged opposing at inlet and outlet of a filter cartridge.

The tubular elastic end coupling, comprises a tube end adapted to fit on the filter cartridge inlet/outlet. The elastic end coupling comprises one or more tubular fluid connections for conveying fluid to and from external means for fluid content separation purposes by said membrane filter cartridge.

### TECHNICAL FIELD

The present invention relates to an assembly of elastic end couplings, connections and a membrane filter cartridge. The invention improves operational design, reduce fabrication time, lower product price, decrease system pressure drop, and insure better performing membrane filter assembly.

### BACKGROUND OF THE INVENTION

Membrane technologies are conventionally used in various separation processes to conduct separations of particles contained in a fluid that may not be technically or economically viable to separate by other technologies. One advantage is that Hollow-Fiber-Filter (HFF) membrane cartridges can be manufactured at a competitive cost compared to ceramic membranes. Competitive cost is though relative and the subject of the present invention.

### PRIOR ART

References is made to:
US20110036764A1 by Yen-Liand Lin titled “Replaceable hollow fiber membrane filter device”.

References is made to larger Hollow Fiber Filter manufactures:
spectrumlabs.com
gehealthcare.com
www.watersep.net
www.parker.com
www.asahikasei.com
www.pall.com

Reference is made to these very informative articles:
“Design and fabrication of hollow fiber membrane modules” by Chun Fend Wan as found in Journal of “Membrane Science” Volume 538, 15 Sep. 2017,Pages 96-107 by Elsevier.
“Manufacturing of Hollow Fiber Membrane” by Venkatesh D. Vandekar are found in international Journal of Science and Research, volume 4 issue 9, September 2015.
“Fabrication of Polymer Membranes” by Volker Abetz, University of Hamburg, Institute of Physical Chemistry, workshop 2018.

### DEFINITIONS RELEVANT FOR THE PRESENT INVENTION

The term “a” or “an” as used herein means one or more or at least one.
The term “autoclaving” used herein is based on pressurized steam to destroy microorganisms, and are the most dependable systems available for the decontamination of laboratory waste and the sterilization of pharma equipment.
The term “backflow” is used to describe a slight harvest return flow, increasing the TMP, Trans Membrane-Pressure somewhat helping high velocity flow along the membrane to remove deposits. Backflow not to be confused with the term “backflush” being widely used in the (rigid) membrane industry though not feasible on elastic membranes such as “hollow fibre module”.
The term “bioreactor” as used herein means a physical device, which biologically active environment is suitable for cultivation of micro-organism performing a desired process suspended in liquid media agitated by an impeller.
The term “broth” as used herein means the non-filtered liquid content, the cultivation soup, the raw fluid in a fermenter or bioreactor consisting of cells, debris, micro-organisms, media with nutrients, waste, harvest, etc. The broth, feedstock, the raw feed enters at inlet and pass the Hollow-Fiber-Filter (HFF) membrane, Cross-Flow-Filter (CFF) membrane whereby the broth becomes slightly concentrated retentate at the CFF exit and as such is returned into and re-mixed with the broth in the bioreactor.
The term “cartridge” as used herein means the functional part of the membrane filter module. The cartridge may consist of a bundle of parallel and longitudinal arranged individual polymeric hollow fibers, lumens, straw inside a polymeric round tube in each end said lumens fixed to the inside of said tube by potting glue. The cartridge becomes a module, unit, assembly when each end of the cartridge is equipped with fittings, connections, end caps, ports, etc. Alternatively, the cartridge consists of porous sheets or flat porous plates stacked next to each other separated by a grid or flow net with an appropriate inlet and outlet.
The term “Cross-Flow-Filter” (CFF) as used herein is a filter device, a separating device which allow a liquid or fluid and in said fluid suspended selectable components to pass the separating device with the liquid volume onto the other side of the device by crossing, passing the device with certain specification eliminating desired suspended components not to pass the separating device. The device has a first entrance for the broth and a second exit for the retentate. In between the broth first entrance and the retentate second exit further a third permeate exit for the product, the filtrate, the harvest which has passed the device.
The term “cultivation” or “culturing” refers to hosting of micro-organism, such as mammalian cells, in a bioreactor for production purposes, such as expression of a product by said micro-organisms or proliferation of said micro-organisms
The term “deposit” or “membrane coating” or “filter cake” or “fouling” as used herein refers to a process where solute, particles, micro-organisms deposit onto a membrane surface, such as a Cross-Flow-Filter membrane surface or even into membrane pores in a way that degrades the membrane's performance and increase transmembrane pressure to undesired levels, Such deposit may be removed by increased shear force such as high velocity of the re-circulating broth for washing purposes.
The term “disposable” refers to a product manufactured often from synthetic materials preferably at low cost and to be scrapped after use. The here presented disposable membrane filter system may be integrated with a Bioreactor System and further bagged and pre-sterilized ready for use.
The term “exterior facility” (area) means laboratory, production facility, testing facility in which room(s) the Disposable Bioprocess System is in use. In the exterior facility also the down-stream process may take place.
The term “filter device” as used here in refer to a Cross-Flow-Filter as mentioned as a CFF or as HFF/Hollow-Fiber-Filter.
The term “fluid” refers to a gas or a liquid, a gas such as air, CO2 or Nitrogen at variable volume or a liquid such as water, broth, solvents or oil at constant volume or a mixture of gases and liquids.

The term “harvest” as used herein refer to the product part (such as a protein) of the broth being the expected product generated by micro-organism being cultivated in a bioreactor or fermented in a fermenter. The harvest (the filtrate, the permeate) may be separated from the broth via membrane and or CFF filtration. When permeate is removed from the CFF the broth becomes concentrated.

The term “hollow fibre module” as used herein refer to a device made from an outer rigid wall tube with end covers housing inside a bundle of thin wall tubes fabricated from porous elastic material such as polyethersulfone or other polymers. The bundle of tubes is sealed, cast into end covers separating the tube inside from the tube outside and hereby appearing as a Cross-Flow-Filter. Port for raw broth, retentate, permeate
The terms “media”, “growth media”, and “nutrient” as used herein are used interchangeable and refers to a sterile complex mixture containing mostly water, carbon sources, various gases such as oxygen and additives such as; vitamins, hormones, growth factors, animal serum, antibiotics, antioxidants, antifoams, cell stabilizers and other components for cultivation of “micro-organisms”. Some media are serum based, some are serum free, animal free, and protein free or chemically defined media. During cultivation the combined media and micro-organism and various debris is named “broth”.
The term “membrane” refers to a boundary layer, which serves as a selective barrier and remains impermeable to specific or desired particles, molecules, or substances when exposed to the action of a driving force (like supplied by a pump). Porous membranes are manufactured from a variety of flexible and rigid materials such as polymers, ceramics and metals, Appear further in the technical term “Cross-Flow-Filter (CFF) device”. The membrane clarifies a part of the broth know to become the harvest.
The term “membrane fouling” as used herein refer to the effect when solids, cells, cell parts, cell membranes, aggregates, etc. creates a layer, a biofilm, a cake of debris on the porous membrane inlet surface. This effect requires different TMP in order to overcome the increased resistance on the combined membrane and deposits and keep if desired constant flux, velocity. Membrane deposits can be removed by high shear forces, high broth velocity and/or backflush.
The terms “micro-organism” or “cells” or “biological cells” as used herein are used interchangeable and is typically divided into: 1. living single-celled organisms, microbes such as; fungus, algae, moss, plankton, yeast, protozoa, eukaryotes, archaea, micro animals, extremophiles and plant cells or the like—2. adherent or semi adherent or suspended living cells such as animal cells, insect cells, mammalian cells, human cells, stem cells—3. prokaryotes and a variety of bacteria such as E.coli or the like—most of the above genetically modified to solve specific tasks and product needs.
The term “module” or “assembly” as used herein refer to the ready to use HFF and consist of the cartridge fitted with the invented elastic end coupling, fittings, fluid connections or traditional end caps in each end.
The term “permeate” as used herein refers to specific parts of a mixture, the broth, a feedstock allowed to pass through a membrane. Also known as filtrate.
The term “permeable membrane” as used herein refer to a porous wall, a “membrane” allowing a liquid and selected particles (size separation) to pass the membrane and certain particles not to pass the porous wall, barrier, separation wall.
The term “ports” as used herein refers to holes, openings anywhere in a wall allowing attachment of suitable fittings, hoses or relevant sensors or general connections selected from the group of; locking ports, press-in ports, or ports involving Luer-Lok fitting, sanitary flanges, sanitary style connections, Tri-clamps, Tri-Clover flanges, connecting fittings, sterile fittings, hoses, tubes, hose barbs, etc.
The term “retentate” as used herein refers to the parts, micro-carriers, particles, “micro-organisms”, debris of a mixture within the broth, feedstock that is held back by a “membrane”, and do not pass the membrane as to its size, shape or charge.
The term “single-use” as used herein refers to a product designed for use only once and to be disposed after use typically delivered “sterilized” and ready to use, such as the “Single-Use-Bioreactor” (SUB) and “Single-Use-Sensors” (SUS).
The term “Single-Use-Bioreactor” (SUB) as used herein refer to a huge variety of flexible wall and rigid wall bioreactors or Single-Use-Fermenter (SUF) preferable pre-installed with one or more “Single-Use-Sensors” all manufactured from disposable materials and sterilized and hereby ready for use eliminating the traditional in-house heat sterilization.
The term “Stainless-Steel” as used herein refers to an alloyed metal based mostly on nickel, chromium, vanadium, carbon, and steel characterized with at least excellent corrosion resistance,
The term “sterilization” as used herein refer to any process that eliminates (removes) or kills (deactivates) all forms of life and other biological agents. Sterilization can be achieved with one or more of the following: heat, autoclaving, chemicals, gases, irradiation, high pressure, and filtration.
The term “sterilized” as used herein refers to a product enclosed in plastic film bags and exposed to sterilization methods which ensures the bag content is sterile. The product is supplied in said film bag(s) to the end-user ready to open and use. The end-user hereby avoids the troublesome heat sterilization, autoclaving similar to classical re-usable equipment processed in an autoclave.
The term “STR” as used herein refers to glass and metal based Stirred-Tank-Reactor used as a bioreactor or fermenter. The STR is not a SUB or SUF.
The term “suspension” or “suspended” as used herein refers to particles, artificial particles, micro-carriers, micro-organism depending on being preferably homogeneous suspended or mobilized in liquid (in the broth) in the STR or SUB or “container”.
The term “Tangential-Flow-Filter” (TFF) as used herein is a device which allow selectable components under pressure to pass from one liquid volume into another liquid volume crossing a “membrane” (Cross-Flow-Filter) eliminating some components to pass the membrane. Such membranes or filter devices may be based on screens, porous material sheets integrated into cassettes or shaped as flat or round plate, tubes, corrugated tubes or stacked hollow fibres into the cartridge.
The term “TMP” or “Trans-Membrane-Pressure” as used herein describes an excellent indicator of membrane fouling. Accumulated micro-organisms, debris, particles on the membrane surface. TMP increases to compensate for the membrane fouling at constant flux.
The term “and/or” as used herein is intended to mean both alternatives as well as each of the alternatives individually. For instance, the expression “xxx and/or yyy” means “xxx and yyy”; “xxx”; or “yyy”, all three alternatives are subject to individual embodiments.

### BRIEF DESCRIPTION OF THE FIGURES

FIG. 1 a and b show the invention of the low-cost membrane assembly consisting of the cartridge and the two elastic end couplings one at each end.

FIG. 2 show one embodiment of the elastic end coupling cut through

FIG. 3 show one embodiment of the elastic end coupling in 3D.

FIG. 4 show cartridge cut through and elastic end coupling illustrating the good flow properties.

FIG. 5 show one embodiment of the elastic end coupling with circular permeate collection volume for easy assembling.

FIG. 6 illustrate one elastic end coupling with cast on sanitary coupling.

FIG. 7 illustrate traditional assembly with the cartridge, two rigid plastics end-caps multiple fitting assemblies, tri-clamps, seals and clamps.

### SHORT PRESENTATION OF THE INVENTION

An assembly for fluid communication with membrane separation device comprising:
a membrane filter cartridge and
an elastic end coupling assembly comprising:
   a plurality of fluid conveying hollow fluid tight hose bodies internally exposed to a fluid said elastic end coupling conveying a fluid between an external means and the two or more fluid conveying hollow bodies of said elastic end coupling
   a hollow hose body raw fluid guide to a cartridge feed inlet end from external means
   a hollow hose body retentate fluid guide from a cartridge exit end to external means
   a hollow hose body permeate fluid guide from a cartridge permeate exit to external collecting means

### Further Embodiments

1. An elastic and/or flexible end coupling assembly (100) for conveying a fluid comprising:
A first elastic and/or flexible hollow fluid tight tubular assembling body (104) adapted for connecting multiple devices conveying multiple fluids, comprising a first hollow fluid conveying connection (107) having an inlet for a raw fluid and a second permeate outlet (109) wherein said first hollow tubular body (104) is adapted to guide feed fluid to a cartridge inlet port, and wherein the second permeate outlet (109) is adapted to guide permeate fluid from a cartridge permeate exit port,
A second elastic and/or flexible hollow fluid tight tubular assembling body (105) adapted for connecting multiple devices conveying multiple fluids, comprising a first hollow fluid conveying connection (110) having an inlet for the retentate fluid and a second permeate outlet (111) wherein said second hollow tubular body (105) is adapted to collect and guide retentate fluid from a cartridge exit port,
A cartridge having a first and a second opening wherein the first tubular assembling body (104) is in fluid tight connection with the first opening and the second tubular assembling body (105) is in fluid tight connection with the second opening.

2. A elastic end coupling assembly (303) according to embodiment 1 for connection to a cartridge (101, 201) said elastic end coupling assemblies comprises:
a first elastic hollow fluid tight tubular assembling body (305) comprises two tubular ring shaped sealing surface areas (535, 536) separated by a fluid collection volume (530) corresponding with said cartridge permeate outlet (230, 231) said fluid collection chamber (530) in fluid communication with a elastic tube part (509)
a second elastic tube part (310) conveying fluids with external means
a third elastic tube part (311, 325) convey the permeate from cartridge (101, 201) permeate outlet (230, 231) to external collection volume (530) and further via hose 509 to external means

said set of elastic end coupling devices (303, 402) connects to either an inlet or an outlet (204, 205) of a cartridge (101, 201, 601).

3. A simplified elastic end coupling assembly (402) according to embodiment 1 and 2 for connection to a cartridge (101, 201) said elastic end coupling comprises:
a first hollow fluid tight tubular assembling body (404) comprises one tubular inner ring shaped sealing surface (432) areas (535, 536)
a second elastic tube part (407) connected to said first hollow fluid tight tubular assembling body (404) for communication of fluids with external means

said simplified elastic end coupling devices (303, 402) connects to either an inlet or an outlet (204, 205) of a cartridge (101, 201, 601).

4. A elastic end coupling assembly according to embodiment 1, 2, 3 comprising a plurality of elastic hoses assembled into said tubular assembling body (104, 105) forming a hollow body manufactured from a variety of polymers predominantly an elastic polymer.

5. An elastic end coupling assembly according to any previous embodiments comprising a plurality of elastic hoses assembled into said tubular elastic end coupling assembly in connection points as an over-moulded assembly.

6. An elastic end coupling assembly according to any previous embodiments comprising further one or more elastic hoses, flanges or connection points with access to the inner fluid conveying volume for connection of various sensors such as pressure, temperature, dissolved oxygen, pH, biomass, mass flow, volume, velocity.

7. An elastic end coupling assembly according to any previous embodiments integrating a thermoplastic elastomeric part such as a Tri-Clamp flange, a Tri-Clamp seal, a tubular part of selectable elastomeric materials, a supporting device casted together with thermoplastic elastomeric parts in an over-moulded process.

8. An elastic end coupling assembly according to any previous embodiments integrating parts such as a Tri-Clamp flange, a Tri-Clamp seal, a tubular part of polymeric materials said supporting devices over-mould casted though bonding or through thermal welding to a thermoplastic elastomeric hollow tubular body part.

9. An assembly for separation specific suspended containments in said through passing raw fluid, according to any previous embodiments, comprising at least one cartridge (101, 201) said cartridge equipped with at least in one end one elastic end couplings (303, 402) manufactured from polymeric materials of single-use character allowing the assembly to be sterilized before use and disposed after use.

10. An assembly for separation of specific containments in raw fluids according to any previous embodiments comprising:
a set of elastic end coupling assemblies comprising
a plurality of fluid conveying hollow fluid tight tubular assembling bodies internally communicating said fluid transmitted between external means and the two or more fluid conveying hollow hose bodies
a hollow tubular bodies guide said raw fluid to said cartridge feed inlet
a hollow hose bodies guide said retentate fluid from cartridge exit to external means
at least one hollow tubular bodies guide said permeate from cartridge permeate exit to external collecting means

said assembly further comprises one or more cartridges (101, 201) mounted with in at least one end (204, 205) said elastic end coupling assembly (303, 402) said cartridge comprising:
a rigid plastic tubular housing (215) forming a fluid-tight barrier separating permeate inside from outside and in at least one end the cartridge forms a fluid-tight cast polymeric sealing cover (216) between the hollow fibers (214) outside circumference and plastic housing wall (215) inside which separates the raw non processed fluids which flows from inlet port through the hollow fiber bundle leaving the cartridge at the opposite exit port end and said
integrated plurality of bundled porous wall hollow fibers (214) said fluid containments separated by the porous wall of said bundled hollow fibers (214) leaving said cartridge (101, 201) as permeate, said
rigid plastic tubular housing comprises a feed inlet (219) said
rigid plastic tubular housing comprises a retentate outlet (220) said
rigid plastic tubular housing comprises a permeate (230, 231) outlet

11. A method of assembling according to any previous embodiments:
a filter cartridge with a raw fluid inlet, retentate outlet and permeate outlet
a first elastic end coupling assembly connected to said cartridge inlet port with at least one mean for fluid tight connection
a second elastic end coupling assembly connected to said cartridge multiple outlet ports with multiple external means for fluid tight connection

establishing a single-use membrane filter device ready for further integration in a process equipment.

### DETAILED PRESENTATION OF THE INVENTION

It is an object of the present invention to provide a price compatible Hollow-Fiber-Filter assembly for separation of specific containments in raw fluids comprising:
a membrane filter cartridge and
an elastic end coupling assembly comprising:
   a plurality of fluid conveying hollow fluid tight hose bodies internally exposed to a fluid said elastic end coupling conveying a fluid between an external means and the two or more fluid conveying hollow bodies of said elastic end coupling
   a hollow hose body raw fluid guide to a cartridge feed inlet end from external means
   a hollow hose body retentate fluid guide from a cartridge exit end to external means
   a hollow hose body permeate fluid guide from a cartridge permeate exit to external collecting means

said elastic end coupling assemblies connected to a membrane filter devise.

### Short Description of the Invention

The invented membrane assembly consists of a cartridge and a set of elastic end-couplings:
1. The cartridge may consist of the bundle of hollow fibers or stacked membrane sheets, the housing tube shell or filter housing and the fluid tight potting in each end of the cartridge/fibers.
2. The elastic end-couplings comprising fluid connections for broth inlet, retentate outlet, and permeate outlet of said cartridge. Said set of elastic end-couplings may have different design and purpose.

### Cartridge Design

Design and fabrication of surface filtration cartridges comprising porous membranes involves different disciplines and requires a thorough understanding of the intended application. There is no such design that serves all needs. The evolution of module design and fabrication is built on a wide range of prior arts and practical experiences.

One design and fabrication of a cartridge comprises a bundle of porous wall hollow fibers inside a round tube.

One other design and fabrication of a cartridge comprising a stack of porous sheets, plates into a cartridge, frame structure.

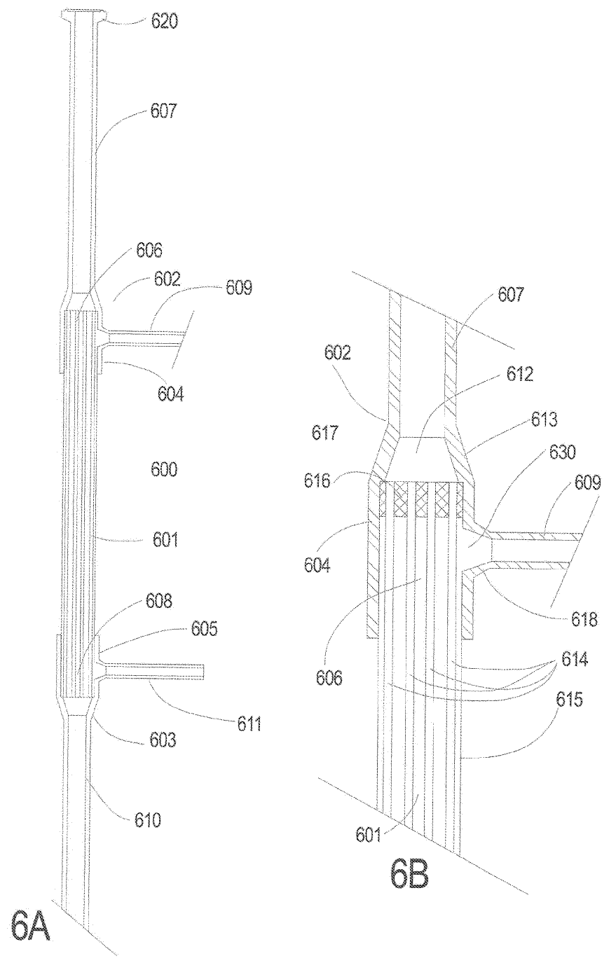

In general, the typical round cartridge has few major parts also illustrated in FIG. 6:
1. the bundle of hollow micro porous fibers
2. the cartridge structural, tubular housing with one cut hole perpendicular to the tube in each tube end (diameter ranging 20-80% of tube housing diameter)
3. the casting, putting wall integrating fluid tight the porous fiber ends in each end of the cartridge tube (putting length 20-100% of tube diameter)

The cartridge is the functional component of the complete HFF assembly. The cartridge consists of numerous tubular membranes of diameter ranging from 0.5-10 mm packed together inside the tubular housing. The packing density is defined as the fraction or percent of the volume in the housing occupied by the Hollow-Fiber membranes. Even the cartridge is symmetrical, identical in both ends said cartridge in practical use will have one feed, broth inlet end and one exit, retentate outlet end.

The hollow fibers are an artificial, organic membranes consisting of a semi-permeable barrier in the shape of a hollow tubular fiber. The multiple pores are arranged perpendicular to the end-less fiber wall. Pores can be arranged asymmetrical, so the pores change size from inner side to outer side of the fiber wall—named asymmetrical design. Wall thickness typical from lees than few tenth of a millimetre to several millimeters.

The hollow fiber membranes divide the cartridge into two compartments or chambers. The inner side of the elongated fiber bundle refers to the space, chamber enclosed by the membrane inlet exposed to non-filtered raw fluid, broth, feed. While the fiber outside refers to the permeate within the collection area. The fiber outside form a chamber between the outer surface of the fiber bundle (separating wall for the raw fluid) and the inner side of tubular housing shell. The housing usually possesses an elongated tubular shape with one or more openings at one or both ends and a uniform circular cross section to minimize manufacturing costs.

At each end of the cartridge a cover 216 insure the open and inner volume of fibers 214 are accessible through the end faces 219, 220. This cover is an agent, a putty, a glue with good adhesion properties and some elasticity after curing such as epoxy or polyurethane or the like. The casted in cover creates a disc with a plurality of holes formed by each of the hollow fibers. The glue surrounds every hollow fiber in a fluid tight seal. The thickness of the cover ranges from a few to 50 mm, most relevant from 5 to 15 mm.

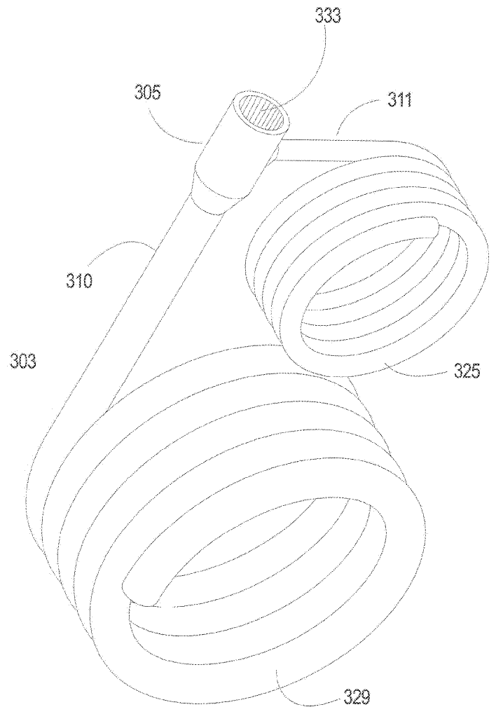

Raw non-processed fluid enters cartridge inlet end face 219 and flows through the inner side of the hollow fibers 214. Each raw fiber inner side extending from end face 219 to end face 220 is to be viewed as inlet to the membrane area. The raw fluid is flowing inside the tube, straw, lumen 214 at a desired velocity. A pressure difference across the tubular membrane wall 214 force a part of the raw non-processed fluid to pass the tubular separation wall 214. Permeate fluid having passed the membrane is collected in chamber 235 and guided to at least one end of the cartridge to exit through port 230 and further into the elastic end-coupling chamber 530, 630. Retentate flow out via cartridge exit, end face 220 for collection by one of the two the elastic end-coupling chambers shown in FIG. 3.

Broth as the un-filtered raw fluid enters in one inlet end of the cartridge and pass through the bundle of hollow fibers along the entire length and exit as retentate in the opposite exit end. Because of a controlled Trans-Membrane-Pressure (TMP) a desired and small portion of the broth pass the porous membrane wall and become the permeate. The permeate, being the desired filtered product, is collected to exit as permeate via a side port in the cartridge housing tubular wall.

Inlet and outlet ports on the housing permit fluid introduction and exit and permeate removal from the shell side of the fiber bundle. In some designs, only one port is needed to collect the permeate from the shell side. The inlet/exit ports on the end cartridge ends permit fluid communication with the fiber lumens.

### Traditional End-Cap Design

Existing Hollow-Fiber-Filter assemblies or modules are flexible membrane fibers housed inside the rigid cartridge housing. Separate rigid plastics or metal end caps connect to the cartridge for various fluid connections. Rigid plastics or metal outer tubing and end-caps is used for various fluid connections.

This traditional end-cap design requires rigid plastic fittings, external fittings, cylindrical tube spacers, conical spacers, clamps including Luer-Lok and Tri-Clamps used broadly in any such industry.

### Traditional Components Description

The typical cartridge consisting of a bundle of parallel and longitudinal arranged individual polymeric hollow fibers, lumens inside a polymeric round tube, housing in each end said lumens fixed to the inside of said tube by potting glue. The tube sheet housing of the cartridge forms a fluid-tight outer seal. The potting glue between fibers and inside housing separates the fluids flowing through the lumen and outside of the lumen behind the housing tube sheet wall.

Housing tube are typical transparent elongated tubular shaped allowing inspection. Diameters ranging from 5 mm up to 250 mm. Length ranging from short 100 mm to long 2.000 mm. Most often the assembly is described in membrane surface area Surface area ranging from cm2 to m2. Housing tube materials are polysulfone or polycarbonate or similar strong and transparent.

The Hollow Fibers (straw, lumen) typical manufactured from polymers like; high density polyethylene (HDPE), polyethersulfone (PES), modified polyethersulfone (mPES), Ultra-high molecular weight polyethylene (UHMWPE), Poly(tetrafluoroethylene) PTFE, Polyvinylidenefluorid (PVDF), cellulose, or ceramics typically via an extrusion process. Even in single material or sandwiched by two or more layers of different materials. Hollow fiber diameter typically ranging from less than 0.5 mm to 20 mm in diameter. Wall thickness typically range from 0.05 to 10 mm. As usual the pore size ranges from Dalton to micro meter (ym). Such as from 50 kD to 1 μm or more.

Optionally hollow fiber membranes manufactured from ceramics such as Alumina oxide, Silicon Carbide, Titanium oxide.

Conventional rigid plastic end-caps, coupling, fittings with ports typical manufactured from polysulfone able to handle autoclavation. Such fittings glued onto the cartridge tube.

Integrated end fittings, end caps, connections come with port such as;
Luer-Lok port—established industrial standard originating from the medical world
Tri-Clamp port—established industrial standard originating from the dairy industry

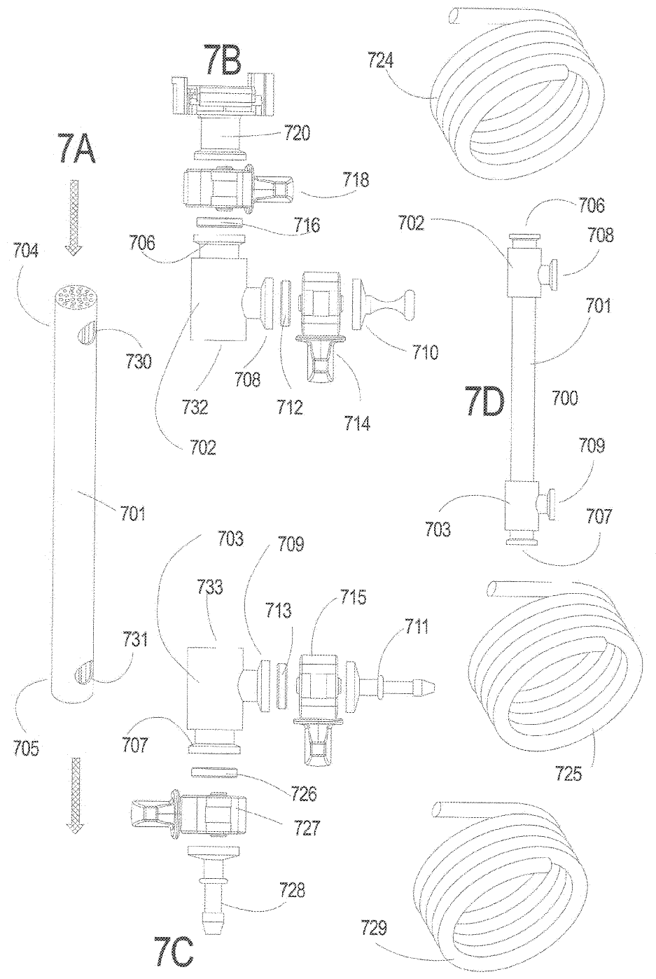

Separate end-fittings, end-caps, connections port couplings is required for further connections. One of the important objects of the present inventions—how to avoid all these expensive extra parts as illustrated in FIG. 7. All those fittings typical manufactured from polycarbonate or from polysulfone for autoclavation requirements. At least 10 manufacturers offer such parts globally,

Further separate aseptic connectors intended for one-time use. In order to assemble parts in laboratory assembling, without parts being autoclaved, the HFF require in each end and permeate outlet one such (expansive) aseptic coupling is required, one clamp, one seal (in total 4 sets).

One clamp is required to connect two sanitary Tri-clamp flanges together around a seal. Clamps is a widely used coupling standard available in both stainless steel and glass fiber re-enforced plastics from many vendors.

Seals, gaskets are required and come in a range of material choices of Silicone, EPDM, PFTE, BUNA N, FKM Fluoroelastorner. Silicone for bio tech application in transparent platinum cured Silicone is most commonly used.

Luer-Lok—a standardized system of small-scale fluid fittings used for making leak-free connections between a male-taper fitting and its mating female part on medical and laboratory instruments. Such as syringes transferring no micro-organisms. Named after the 19th century German medical instrument maker Hermann Wülfing Luer and now defined in the ISO 594 standard. Luer-Lok male connection has an fixed inner diameter of 3.2 mm and for many biomedical applications with low volume flow not a limitation. But for medium flow application in the biotech industry a serious limitation where the high fluid velocities cause severe shear forces killing micro-organism being conveyed and processed.

In general, all the Tri-Clamp fittings, tube parts, clamps, etc. design originate from the dairy industry almost 100 years ago. Tri-Clamp became the brand since 1919 best known from Tri-Clover-Machine Co. in USA. Metal Tri-Clamps are now owned by AlfaLaval. Tri-Clamps are defined in standard ISO-1127 from 1996 and DIN-32676 among others. Sizes range from ¾″ to more than 4″. Flow properties, laminar flow was not of concern and parts design originate from what sheet metal, metal banks, metal rods were available for easy machining and welding. Exactly the same dairy stainless-steel dairy design has continued into the biotech industry with now injection moulded plastic parts. But for the pharma industry flow properties, laminar flow, dead volumes are of outmost importance to avoid.

### Description of Usage

The total assembly is often designed to be all single-use and as the HFF cartridge is integrated with the Single-Use-Bioreactor also the HFF is of single-use design. The custom designed elastic rubber body is mounted on each of the housing shell ends said rubber body facilitate two sealing area on the housing shell circumference. The first seal area is behind the permeate housing shell wall outlet the second seal area is in front of the permeate housing shell wall outlet. Hereby supplying a tight permeate outlet. The second seal area is in front of the permeate housing shell wall outlet further insure a fluid tight broth inlet and retentate outlet. Said broth inlet and retentate outlet is via the integrated hoses in connection with external means and as to such operating under sterile conditions inside elastic rubber body and inside the Hollow-Fiber-Filter module housing shell.

Permeate exit is a suitable in size cut hole in the rigid plastic housing wall close to at least one end of the rigid plastic wall before the putting glue secure fiber ends fluid tight end faces inside each of the cartridge ends.

Further assembling requires the invented silicone elastic end-couplings is pressed in over the end of the cartridge housing shell. As to the elastic end-couplings elasticity fluid tightness is obtained and tightness around the permeate exit hole in the tubular cartridge wall as well as the cartridge tube ends accessing the fiber bundle. Further fluid tightness may be obtained by adding plastics or metal clamps outside the elastic end-couplings.

In small scale bioreactor application, the rigid plastic HFF housing tube is equipped with rigid plastics end fittings, end caps, connections with port. These HFF ports are most often of type Luer-Lok or the larger sanitary flange type. Further connection required yet sets of Luer-Lok or sanitary flanges equipped with barbs for connection to elastic tubing. A costly construction which add unwanted extra internal dead volume, fluid length, very different broth velocities, complexity to keep liquid tight, and in specific add to system pricing. For various cultivation in the pharma industry the product is separated from the micro-organisms containing broth. Its very important to keep internal dead volume, fluid length to a minimum. Its here that the micro-organisms find low velocity media areas to accumulate and die.

### FEATURES TO THE INVENTION

Reality is that the rigid plastic Hollow-Fiber-Filter round cartridge, housing only need the invented elastic hose connections for the transport of fluids to and from. One hose for broth inlet, one for retentate outlet and one or two for permeate recovery. The many end caps, ports, connections are not needed for operation and only increase weight, complexity, possible leaks, physical size and product cost significantly.

The present invention eliminates all the expensive coupling parts and reduce significantly unwanted extra internal dead volume by adding the elastic hoses directly to the rigid plastic cartridge housing. The elastic T-design of the elastic end-coupling assembly insure simultaneously tight connection for: 1. broth inlet and: 2. retentate outlet and: 3. permeate exit—all by the invented elastic assembly connected to the cartridge.

Materials such as rigid polymers to form one or more parts of the invention are, but not limited to materials such as; polycarbonates, polyesters, nylons, polyimide, PTFE resins and other fluoropolymers, acrylic and methacrylic resins and copolymers, polysulphones, polyethersulphones, polyarylsulphones, polystyrenes, polyetherimides, polyethylene terephthalates, polyvinyl chloridcs, chlorinatcd polyvinyl chloridcs, ABS and its alloys and blends, polyolefins, preferably polyethylenes such as linear low density polyethylene, low density polyethylene, high density polyethylene, and ultrahigh molecular weight polyethylene and copolymers thereof, polypropylene and copolymers thereof and metallocene generated polyolefins.

Materials elastic suitable for manufacturing the invented elastic end-couplings are medical grade polymeric materials and elastic polymeric materials. In general Silicones are excellent for the purpose. Relevant groups of Silicone materials are among many Elastosil Silicone from Wacker. Hoses made from PVC is also relevant. Combinations of different materials by various gluing or welding methods. Such as a rigid plastic Tri-clamp and the elastic hose part.

Methods for shaping, casting, moulding with polymers are via the pressure cast/curing of one component RTV silicone. Extrusion moulding of Silicones benefit from dual compound Silicones.

Pressure casting or injections moulding give advantage to a free design of the internal shape with focus on laminar flow properties, controlled fluid velocities and elimination of dead volumes. As to such broth velocities are kept identical all over the fluid cross section and hereby advantageous for the micro-organisms living in the media.

Injection moulding or press moulding of the elastic end coupling allow a choice of integration of multiple elastic and/or elastic or rigid plastic connections or barbs or fittings for different purposes.

An elastic end coupling may integrate a rigid injection moulded thermoplastic part such as a Tri-Clamp flange or a tube part. Alternatively, elastomeric Tri-Clamps flanges, Tri-Clamp seal, a tubular part of selectable elastomeric materials, a supporting device may be cast together with thermoplastic elastomeric part in an over-moulded process.

Such as add on, connections with Single Use Pressure Sensors as offered by American company PendoTech or ScriLog from Parker. Standard available with barb, Luer-Lok and Tri-Clamp connections.

The choice of elastic hoses for inlet and exit allow use of clamp-on flow sensors. Such as LeviFlow offered by Swiss company Levitronics. Or the German Em-Tec clamp-on-sensor or similar from SonoTec. This type of sensors benefits from the elastic tube to measure flow, velocity inside and its flexibility for installation, clamp-on procedure.

Further benefit of the invented elastic end coupling assembly is the free choice of integrated multiple elastic and selectable length hoses connecting by welding. Very often used in the biopharma industry to connect two silicon hoses of the same diameter via welding. Such tube welding can be done sterile with equipment from Terumo, Saint-Gobain, AdvantaPure, etc.

With a hose fully integrated Tri-Clamps, Y or X connections is named over-moulded. Such single-use moulded manifolds and connections provide a seamless internal transition between tubing and connections for a continuous, leak-proof flow. Moulded connections include Y, T, cross, reducer, Tri-Clamp®, and mini Tri-Clamp styles. Over-moulded Tri-Clamp ends greatly reduce bioburden entrapment situations associated with conventional barb-type fittings. They integrated parts of the present invention cost significant less than rigid plastics counterparts or manual mount, making the invented design an attractive option for pharmaceutical, medical, surgical, food, beverage, etc.

The invented elastic end coupling assembly and membrane filter assembly is suitable for applications involving aseptic transfer, sterile processing, and other critical applications in the pharmaceutical, biopharmaceutical, and high purity industries in general. The assemblies reduce manufacturing cost, installation time, improve cleanliness, and retain the many benefits of silicone.

The invented elastic end coupling assembly facilitates optionally more than one integrated cartridge connection. Cartridge elastic end coupling with multiple connections allow multiple cartridges obtaining specific advantages. Such as filter area alteration, length modification keeping filter surface area constant. Or multiple cartridges of different housing dimensions and specifications.

### DESCRIPTION OF FIGURES

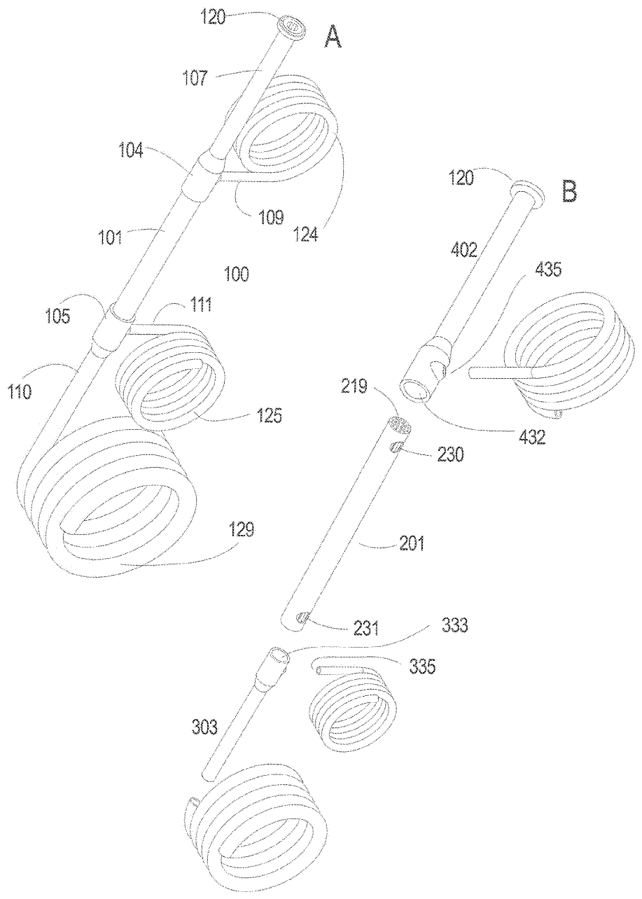

FIG. 1a show one embodiment of the invention of the membrane assembly 100 comprising the cartridge 101 equipped with two elastic end-coupling assemblies 104, 105 one at each cartridge 101 two ends. Its seen that the elastic end-couplings 104, 105 integrates with various hoses 107, 109, 110, 111 for simple fluids exchanges with external devices fully without any expensive fittings, seal, connectors, couplings, etc. (obviously not shown!). Grey raw fluid from an external circuit, container, pump to be processed enter (alternatively through integrated Tri-clamp flange 120) via inlet hose 107 to hollow round tube section 104 chamber and directed to the cartridge inlet 101. Optional one first permeate outlet via exit 109 and long hose 124. Cartridge 101 process the incoming grey raw fluid (broth) into permeate the desired product and the retentate waste. Optionally one second permeate outlet via exit hose 111 and connection hose 125. Retentate from cartridge exit 101 and enter chamber inside the elastic end-couplings 105 guided through and into exit hose 110 and long connection hose 129 back to grey, raw fluid circuit, container.

FIG. 1b show exploded embodiment of the invention illustrating the remarkable simplicity in that the complete assembly consist of only 3 components (201, 303, 402) (ref to FIG. 7).

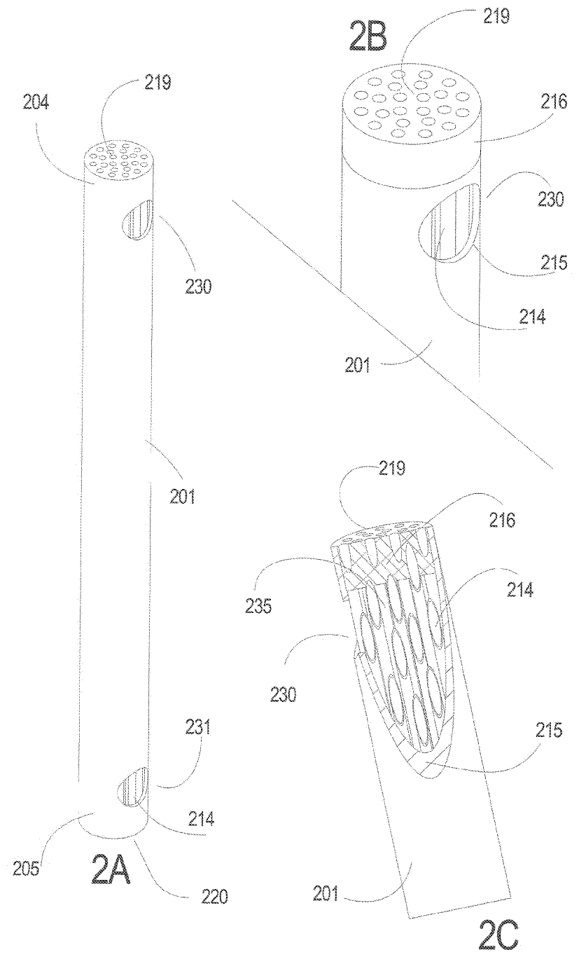

FIG. 2 show one embodiment 2A of the cartridge body 201 with a first inlet end 204 and inlet face 219 and opposite cartridge end 205 and outlet face. Each cartridge 201 end 204, 205 of the body 201 facilitate two radially facing permeate exit hole 230, 231 with direct access to the harboured, bundled fibers 214 outside surface and permeate collection chamber 235.

FIG. 2B show the multiple fibers 214 bundled inside the cartridge 201 plastic tube with a wall 215 and identically in each end secured mechanically suspended between putting cover volume 216 glued fluid tight to each fiber outer surface by adhesion with the putting cover 216 material. Each individual hollow fiber 214 is on all its circumference in contact with the putting material for fluid tight sealing.

FIG. 2C in particular show permeate outer side of the bundled hollow fibers 214 and inside of housing wall 215 and in between the two putting covers 216 creating the collection chamber 235 for processed fluids.

FIG. 3 show one first fluid exit embodiment 303 of the invented elastic end-coupling combining three fluid conveying hollow tube parts together into one hollow embodiment 303. One first fluid conveying hollow round tube section 333 of 305 to be fitted over the cartridge (601, 701) end circumference creating collection chamber 613. One second fluid conveying hollow tube 310 extending axially away 329 from round tubular part 305 for retentate fluid outlet from cartridge (601, 701). Radially extending from round tubular part 305 defining the collection chamber the permeate fluid exit tube 311 is moulded with, connected 335 to a selectable length tube 325 conveying permeate to an external collection point.

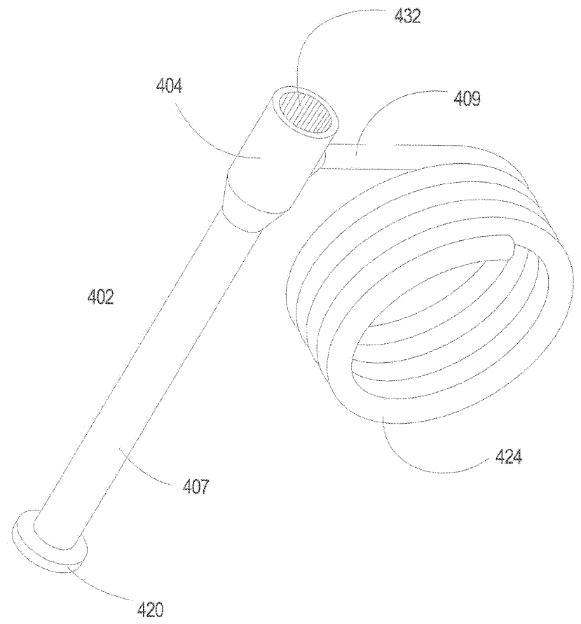

FIG. 4 show one second fluid inlet embodiment 402 of the elastic end-coupling used for the inlet application to the cartridge (601, 701). The tubular hollow embodiment part 404 with opening 432 fit fluid tight over the cartridge inlet end (504, 704) hereby creating an inlet collection chamber 534. Optional permeate outlet hose 409 of a suitable length 424 moulded in connection point 435 to hollow embodiment part 404. Fluid broth inlet hose 407 insure fluid access from external source via sanitary Tri-clamp flange 420 (120) to tubular hollow embodiment 404.

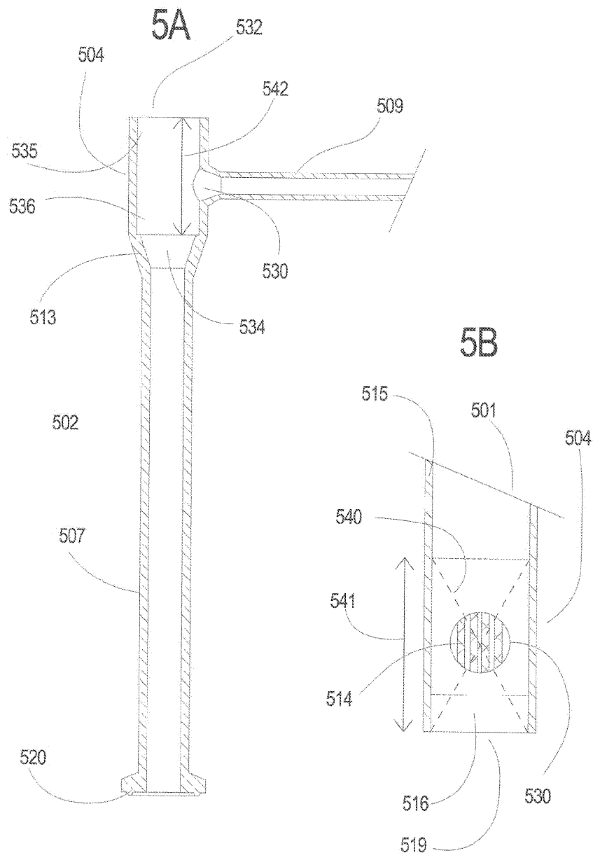

FIG. 5 show one embodiment 502 of the elastic end-coupling cut through lengthwise at 5A. The inlet elastic end coupling 502 with tubular part 504 matching inner diameter for a fit over the cartridge outer diameter and end face 519 along the 541 length of cartridge 501. The tubular part 504 comprises further a fluid collection volume 534 connected to a hose part 507. Opposite end of hose 507 optional integrated over moulded sanitary Tri-Clamp flange 520 for external broth supply device connection (such as a pump). Permeate outlet hose part 509 conveying the harvest and are of selectable length and of small inner diameter optional at opposite end of hose a connector not shown.

Inlet chamber 534 surrounded by funnel 513 (613) between inlet hose 507 and tubular part 504 guides the feed, broth from inlet at Tri-clamp flange 520 to cartridge body 501 inlet face 519.

For a fluid tight connection between cartridge 501 and tubular part 504 a circular area of specific awareness is created. These areas 535, 536 is part of the tubular part 504 and intended for compression by external means to secure tightness. Optional on the external surface of tubular part 504 extra re-enforcement may be added. Such a strips, bands, clamps made from Nylon or metals.

At figure part 5B the cartridge 501 (601, 701) facilitate through the plastic tube housing wall 515 permeate outlet hole 530 (not shown second hole (730, 731)) of the tubular body to which the inlet elastic end coupling 502 tubular part 504 and permeate inlet 518 matches avoiding to restricting permeate fluid exiting hole 530.

Shown with a dashed cross of two lines 540 the cartridge end surface area and with arrow 541 the approximate length from cartridge 501 end face 519 equivalent to internal length 542 of tubular part 504. Almost identical for both cartridge 501 ends.

The inlet elastic end coupling 502 inlet opening 532 for the cartridge 501 (701) enclosed fluid tight around the cartridge 501 end surface 504.

FIG. 6 illustrate one embodiment 6A of the membrane filter assembly invention 600 with cartridge 601 integrating the two elastic end couplings 602, 603. The assembly is cut half through lengthwise for expletory purposes.
One first inlet elastic end coupling, 602 with tubular part 604 is press mounted over cartridge 601 end part 606. The tubular part 604 equipped with integrated hose part 607 and integrated silicone sanitary Tri-Clamp flange 620 for broth supply device connection, permeate outlet hose part 609 of selectable length selectable opposite hose end connector not shown—hose could very well be delivered closed.
One second exit elastic end coupling 603 with tubular part 605 is press fit mounted over cartridge 601 end part 608 equipped with integrated silicone hose 610 of selectable length and diameter, permeate outlet hose part 611 of selectable length and possible connector—opposite end of hose delivering permeate the harvest, product receiving device not shown.

Expanded sectional part 6B of complete assembly 6A part show details of the invention. The assembly is cut half through lengthwise for explanatory purposes. Of the inlet elastic end coupling 602 the tubular part 604 is press mounted over cartridge 601 cylindrical end part 606. The cartridge 601 consist of the bundle of hollow fibers 614 (four shown) inside the plastic tube housing wall 615 assembled in inlet end (outlet end as well) with putty 616 insuring ail fibers 614 are mechanical hold in place and insure a fluid tight separation of inner and outer fiber 614 surfaces.

Inlet elastic end coupling, 602 extent from its cylindrical tube part 604 to a funnel 612 with conical funnel walls 613 to insure identical velocity (laminar flow) calculated of inlet tube 607 inner cross section to the bundled fibers 614 total cross section (fiber 614 number×fiber inner cross section area).

The permeate outlet hole 630 on cartridge 601 rigid plastic tubular housing wall 61 to be aligned with the permeate receiving inlet funnel 618 in front of permeate hose 609 (or hole 731 aligned up with hose barb 711 at exit).

The illustrated invented membrane filter assembly consist of only 3 individual parts!

FIG. 7 illustrate the commercially available and traditional membrane filter assembly (Hollow-Fiber-Filter (HFF)) setup. In sketch part 7B and 7C all parts shown in exploded view. The commercially available membrane filter assembly 700 is further show in reduced size in 7D. The arrows shown at 7A show fluid direction.

The membrane filter assembly 700 (Hollow-Fiber-Filter (HFF)) is only available as the assembly 700 consisting of the raw cartridge 701 integrated with, glued on traditional rigid plastics end-caps 702, 703 in both ends of cartridge 701 creating inlet port 706 and outlet end port 707 as well as permeate exit ports 708 and 709.

Each rigid plastic end-cap 702, 703 equipped with two tri-clamp flanged ports 706, 708 and 707, 709, For end-cap 702 axial broth inlet 706 and radial exit 708. For end-cap 703 axial retentate exit 707 and radial permeate exit 709. Of the dual radial exit ports 708, 709 for the filtered product, the permeate typically only one port is used 709 and the radial port 708 closed or vice versa. The clamp 714 holds blocking-plate 710 tight via the seal 712 to tri-clamp flange on port 708. Its worth to notice that all 4 ports on the two rigid plastics end-caps 702, 703 facilitate Tri-clamp flanges.

At membrane filter assembly 700 rigid plastics inlet end-cap 702 axial port 7 06 is equipped with various and necessary fittings: silicone ring seal 716, Tri-clamp flange aseptic connector 720, clamp 718 and possible further one tri-clamp aseptic connector (not shown) and hose 724 connection.

At membrane filter assembly 700 axial end-cap 703 the retentate exit port 707 following parts is needed; silicone ring seal 726, sanitary flange barb connector 728, clamp 727, hose 729 mounted on barb 728.

Permeate outlet radial port 709 is open exit via ring seal 713, sanitary flange 1 barb connector 711 secured with clamp 715 to be mounted with hose 725,

Cartridge 701 have two housing permeate outlets 730 one in each end of the cartridge with access to the fibers outside surface and permeate collection volume.

Standard available membrane filter assemblies consist of >18 individual parts. Significantly adding to assembling time and cost of the membrane filter assembly.

While the present invention has been described in connection with the particular embodiments thereof, it will be understood by those skilled in the art that many changes and modifications may be made without departing from the scope of the invention as defined by the appending claims.

1-12. (canceled)

13. A clamp-free and flange-free fluid tight assembly element for conveying and filtering a fluid comprising:
a) a first hollow fluid tight tubular wall body having a first opening and a second opening located in each end of the first hollow fluid tight tubular wall body, and comprising a third opening located in the wall body,
b) a second hollow fluid tight tubular wall body having a first opening and a second opening located in each end of the second hollow fluid tight tubular wall body, and comprising a third opening located in the wall body, and
c) a third hollow fluid tight tubular wall body comprising a filter, the body having a first opening and a second opening located in each end of the third hollow fluid tight tubular wall body, and having a third opening located in the wall body adjacent the first opening and a fourth opening located in the wall body adjacent the second opening;
wherein the first opening of the first hollow fluid tight tubular wall body communicates with the first opening of the third hollow fluid tight tubular wall body, the second opening of the first hollow fluid tight tubular wall body is adapted to receive the fluid, and the third opening of the first hollow fluid tight tubular wall body communicates with the third opening of third hollow fluid tight tubular wall body,
wherein the first opening of the second hollow fluid tight tubular wall body communicates with the second opening of the third hollow fluid tight tubular wall body, the second opening of the second hollow fluid tight tubular wall body is adapted to guide the filtered fluid, and the third opening of the second hollow fluid tight tubular wall body communicates with the fourth opening of third hollow fluid tight tubular wall body.

14. The element of claim 13, wherein the filter is a membrane filter.

15. The element of claim 13, wherein the first and second hollow fluid tight tubular wall bodies are elastic.

16. The element of claim 13, wherein the fluid is a broth.

17. The element of claim 13, wherein the third opening of the first and second hollow fluid tight tubular wall body are in fluid communication with an elastic tube part.

18. The element of claim 13, wherein the third opening of the first and second hollow fluid tight tubular wall body are in fluid communication with a volume defined by an extension of the third opening and the inner volume of the first and/or third tubular wall body.

19. The element of claim 13, wherein the first and/or second hollow fluid tight tubular wall body comprises a tubular ring-shaped sealing surface on the inner side of the wall body.

20. The element of claim 13, wherein the second opening of the first hollow fluid tight tubular wall body and/or the second opening of the second hollow fluid tight tubular wall body is in fluid communication with an elastic tube part.

21. The element of claim 13, wherein the element is manufactured from a polymer, preferably an elastic polymer.

22. The element of claim 13, wherein the connection points between the first hollow fluid tight tubular wall body and the third hollow fluid tight tubular wall body and the connection points between the second hollow fluid tight tubular wall body and the third hollow fluid tight tubular wall body are assembled in an over-moulded assembly.

23. The element of claim 13, wherein the third hollow fluid tight tubular wall body is selected from a hollow fiber membrane filter, such as a HFF or CFF.

24. A hollow fluid tight tubular wall body having a first opening and a second opening located in each end of the first hollow fluid tight tubular wall body, and comprising a third opening located in the wall body, for use as the first and/or second hollow fluid tight tubular wall body in the clamp-free and flange-free fluid tight assembly element of claim 13.
